(19) United States
(12) Patent Application Publication
(10) Pub. No.: US 2025/0161072 A1
(43) Pub. Date: May 22, 2025

(54) SACRO-ILIAC JOINT STABILIZING IMPLANTS AND METHODS OF IMPLANTATION

(71) Applicant: SI-Bone Inc., Santa Clara, CA (US)

(72) Inventors: W. Carlton RECKLING, Cheyenne, WY (US); Scott A. YERBY, Montara, CA (US); Paul M. SAND, Redwood City, CA (US)

(21) Appl. No.: 18/809,229

(22) Filed: Aug. 19, 2024

Related U.S. Application Data
(63) Continuation of application No. 17/116,903, filed on Dec. 9, 2020, now Pat. No. 12,083,026.
(60) Provisional application No. 62/945,653, filed on Dec. 9, 2019.

(51) Int. Cl. A61F 2/46; A61B 17/16; A61B 17/17; A61B 17/56; A61B 17/70; A61B 90/00; A61F 2/30

(52) U.S. Cl. CPC A61F 2/4611; A61B 17/1662; A61B 17/1739; A61B 17/7055; A61F 2/4603; A61B 2017/564; A61B 2090/376; A61F 2002/30995; A61F 2002/4687

(57) ABSTRACT

Methods of implanting SI joint stabilization implants across a SI joint from a dorsal approach. The methods may include advancing an elongate implant positioning guide in a dorsal trajectory into an ilium of a subject, directly engaging a guide interface member of a SI joint stabilization implant with the positioning guide to restrict movement of the implant with respect to the positioning guide in at least one direction, and advancing the implant across the SI joint while guiding the implant with the positioning guide.

### CROSS REFERENCE TO RELATED APPLICATIONS

This application is a continuation of U.S. patent application Ser. No. 17/116,903, filed Dec. 9, 2020, which claims the benefit of U.S. Provisional Application No. 62/945,653, filed Dec. 9, 2019, the entire disclosures of which are fully incorporated by reference herein for all purposes.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Implants may be positioned across a sacro-iliac (“SI”) joint to help stabilize the joint. Regions of the ilium may have greater density than regions of the sacrum. Depending on one or more of the delivery trajectory, the target location for implantation, and the configuration of the implant, the differences in bone density may present challenges while advancing some SI joint implants across the SI joint. Implantation methods are needed that accommodate for the differences in bone density and can facilitate the successful delivery of the SI joint implant from a dorsal approach.

### SUMMARY OF THE DISCLOSURE

One aspect of the disclosure is a method of positioning a SI joint stabilization implant across a SI joint from a dorsal approach. The method may include advancing an elongate implant positioning guide (“positioning guide,” or “guide”) in a dorsal trajectory into an ilium of a subject, engaging a guide interface member of a SI joint stabilization implant with the positioning guide to restrict movement of the implant with respect to the positioning guide in at least one direction, at a time subsequent to the engaging step, advancing the implant across the SI joint while guiding the implant with the positioning guide, and removing the positioning guide from the ilium.

In this aspect, the engaging step may limit the amount of implant migration towards the sacrum while advancing the implant across the SI joint.

In this aspect, advancing the positioning guide into an ilium may comprise advancing the positioning guide into the ilium between lateral and medial cortical walls of the ilium. In this aspect, advancing the positioning guide may comprise advancing the positioning guide only into the ilium.

In this aspect, the method may optionally further comprise, at a time subsequent to positioning the positioning guide into the ilium, advancing a sacrum positioning guide into a sacrum of the patient, engaging a second guide interface member of the implant with the sacrum positioning guide, wherein the implant advancing step occurs while also guiding the implant with the sacrum positioning guide, and removing the sacrum positioning guide from the sacrum. Engaging a second guide interface section of the implant with a sacrum positioning guide may occur prior to advancing a sacrum positioning guide into a sacrum of the patient. The method may further include advancing a second sacrum positioning guide in a dorsal trajectory or dorsal approach into the sacrum of a subject, and engaging a second sacrum guide interface member of the implant with the second sacrum positioning guide to restrict movement of the implant with respect to the second sacrum positioning guide in at least one direction.

In this aspect, the method may optionally further include, at a time prior to engaging the guide interface member with the positioning guide, engaging a sharpened broach with the positioning guide, and advancing the broach towards the SI joint to create a space for the implant while guiding the broach with the positioning guide, and removing the broach to allow dorsal access to the space.

In this aspect, engaging a guide interface member of the implant with the positioning guide may comprise positioning an annular member of the implant over a proximal region of the positioning guide.

In this aspect, engaging a guide interface member of the implant with the positioning guide may comprise positioning an arcuate member of the implant around a proximal region of the positioning guide.

In this aspect, the method may further comprise advancing a second ilium positioning guide in a dorsal trajectory into the ilium of a subject, and engaging a second guide interface member of the implant with the second ilium positioning guide to restrict movement of the implant with respect to the second ilium positioning guide in at least one direction.

One aspect of this disclosure is a method of implanting a SI joint implant across a SI joint from a dorsal approach while preventing the SI joint implant from migrating away from the ilium and towards the sacrum. This aspect may include any other method step or sequence of steps claimed and/or described herein. In this aspect, the preventing step may include engaging a positioning guide with a portion of the implant.

### BRIEF DESCRIPTION OF THE DRAWINGS

FIGS. 1A and 1B illustrate an exemplary SI joint implant engaged with a positioning guide.

FIGS. 2A and 2B illustrate an exemplary SI joint implant engaged with first and second positioning guides.

FIGS. 3A and 3B illustrate an exemplary SI joint implant engaged with a positioning guide.

FIGS. 4A and 4B illustrate an exemplary SI joint implant engaged with first and second positioning guides.

FIGS. 5A and 5B illustrate an exemplary SI joint implant engaged with a plurality of positioning guides.

FIGS. 6A and 6B illustrate an exemplary SI joint implant engaged with a positioning guide.

FIG. 7 illustrates an exemplary SI joint implant.

FIG. 8 illustrates a portion of an exemplary SI joint implant engaged with a positioning guide.

FIG. 9 illustrates a portion of an exemplary SI joint implant engaged with a positioning guide.

FIG. 10 illustrates an exemplary SI joint implant engaged with a plurality of positioning guides.

FIG. 11A illustrates a posterior view and exemplary locations for positioning guides.

FIG. 11B illustrates an exemplary implantation location for a SI joint implant across a SI joint.

### DETAILED DESCRIPTION

The disclosure herein is related to SI joint stabilizing implants and methods of implanting SI joint stabilizing implants across a SI joint. The methods include implanting a stabilizing implant from a dorsal approach across the SI joint with a first portion of the implant positioned in the ilium and a second portion of the implant positioned in the sacrum. The implants herein are sized and configured to be implanted according to any of the methods of implantation herein, unless indicated to the contrary.

Regions of the ilium into which a portion of the implant is positioned may have greater density than regions of the sacrum into which a second portion of the implant is positioned. When positioning a SI joint implant across a SI joint from a dorsal approach, the implant may tend to deflect away from denser cortical iliac bone and migrate towards and into the less dense sacrum, preventing proper positioning of the implant across the SI joint. Implantation methods and implants are described herein that can maintain proper implant trajectory when advancing the SI joint implant across the SI joint from a dorsal approach. The methods and approaches herein can account for the differences in bone density between the ilium and sacrum and prevent the implant from migrating away from denser iliac bone during implantation.

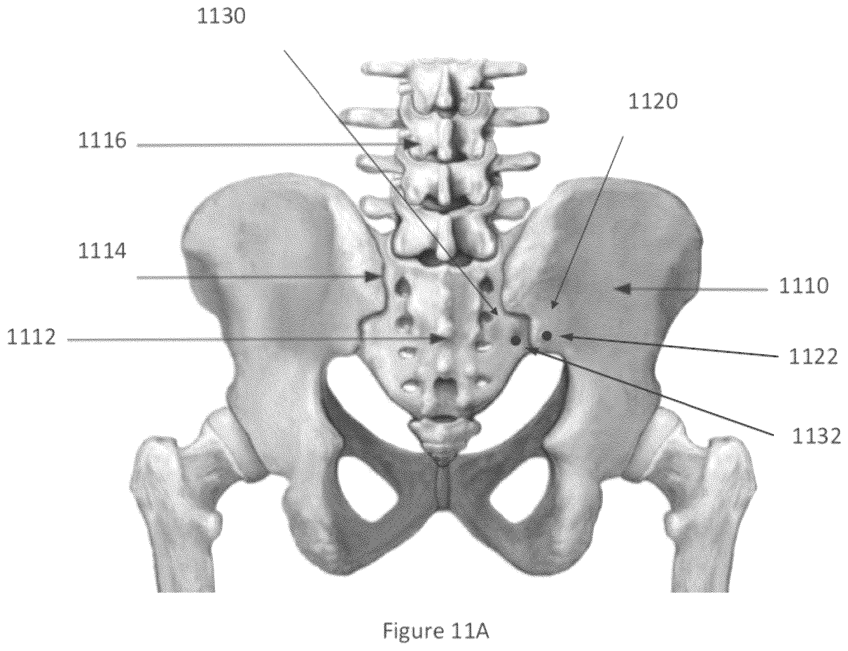

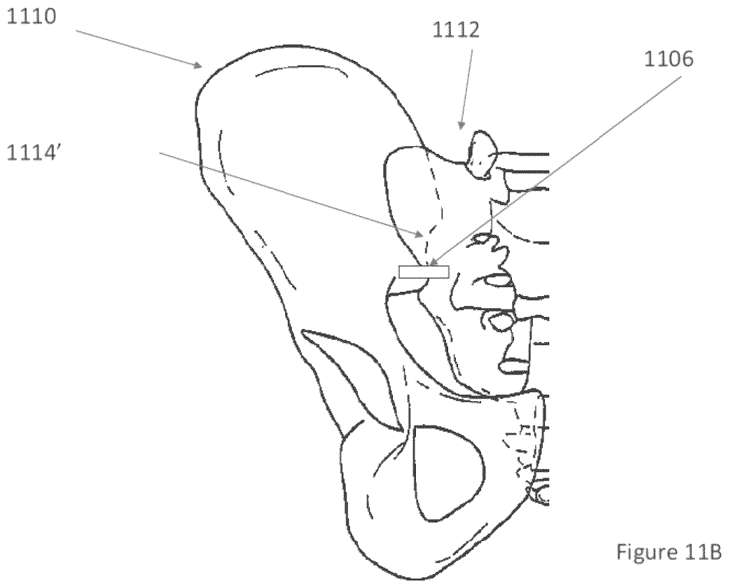

The methods herein may include advancing one or more positioning guides, any of which may be referred to herein as a “guide,” into an ilium from a dorsal approach, and in some embodiments between lateral and medial cortical walls of the ilium. FIG. 11A illustrates a posterior view and a general dorsal approach for implanting the SI joints herein across the SI joint. FIG. 11B illustrates an exemplary implant 1106 implanted across a SI joint 1114′ with a first region of the implant disposed in ilium 1110, a second region of the implant disposed in sacrum 1112, and a central region extending across the SI joint 1114′. FIGS. 11A and 11B, which are referenced in more detail below, illustrate ilium 1110, sacrum 1112, the SI joints 1114 and 1114′, and lumbar vertebrae 1116. FIG. 11A also illustrates a general region 1120 for a starting point for advancing an ilium positioning guide into the ilium, and a general region 1130 for a starting point for advancing a sacrum positioning guide into the ilium. FIG. 11A further illustrates an exemplary ilium starting point 1122 for an ilium positioning guide, as well as an exemplary sacrum starting point 1132 for a sacrum positioning guide. Any of the ilium positioning guides herein may have a starting point in general region 1120, such as ilium starting point 1122. Any of the sacrum positioning guides herein may have a starting point in general region 1130, such as sacrum starting point 1132. A radiographic view image may be obtained and utilized to help guide the positioning guide into the ilium between lateral and medial cortical walls of the ilium, which are illustrated generally in FIGS. 11A and 11B. Methods herein include interfacing the ilium positioning guides herein with a portion of the SI joint implant, such as an interface member of the implant, to guide the implant across the SI joint. By positioning an ilium positioning guide in the relatively dense region of the ilium, and interfacing and engaging the positioning guide with the implant, the guide can help ensure a portion of the implant will stay on course during advancement during implantation in the dorsal approach, rather than migrating away from the relatively dense cortical ilium bone and towards the sacrum. The positioning guides herein thus interface directly with the implant and are sized and configured to act as a guide for a portion of the implant to ensure that the ilium portion of implant is properly positioned in the ilium and that the implant is properly implanted across the SI joint.

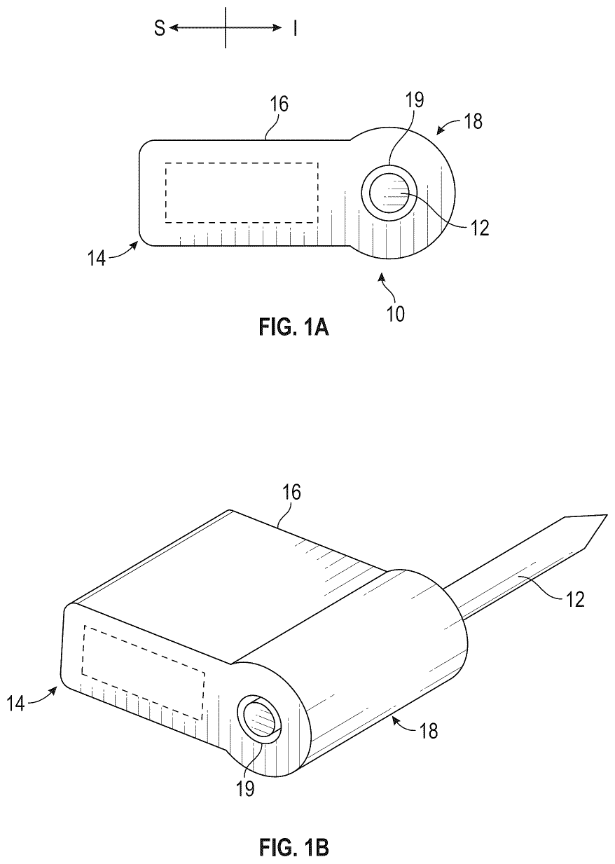

The positioning guides are sized and configured to generally restrict movement of the implant with respect to the positioning guide in at least one direction. The implant may be free to move relative to the positioning guide in other ways or directions. For example, once interfaced, the implant may still be able to rotate relative to the guide, such as in FIGS. 1A and 1B, but the guide can still maintain the desired trajectory of at least a portion of the implant when the implant is advanced in the dorsal trajectory over the guide.

The methods herein include advancing the implant across the SI joint, while the guide helps guide a portion of the implant into the ilium. The methods may also include removing the positioning guide from the ilium after the implant has been positioned across the SI joint.

The methods herein may include positioning more than one positioning guide, optionally more than one guide in the ilium, and optionally one or more guides into sacral bone. Any of the one or more guides herein may be sized and configured to function as a positioning guide to help guide a portion of the implant into ilium bone or sacral bone.

In some alternative methods and implants, it may be possible to advance a SI joint stabilizing implant from a dorsal approach across a SI joint without using a positioning guide. For example, these methods may include radiographically visualizing a teardrop view of the ilium and advancing the implant while visualizing the teardrop view to ensure a portion of the implant stays sufficiently on course into the teardrop region of the ilium. Any of the methods herein may thus optionally exclude an ilium positioning guide, and may rely on a radiographic image, such as a teardrop view, to help maintain a desired implant trajectory into a teardrop region of the ilium. Implants implanted according to these methods may be implanted with or without a broach (described in more detail below), and if implanted without the use of a broach, the implants may have distal end regions that are configured to penetrate into bone, optionally having sharpened distal ends.

The SI joint stabilizing implants herein, such as any of those shown in FIGS. 1A-10, are generally sized and configured to be able to interface with an elongate ilium positioning guide, and may be sized and configured to interface with one or more additional positioning guides, which may be ilium or sacral guides.

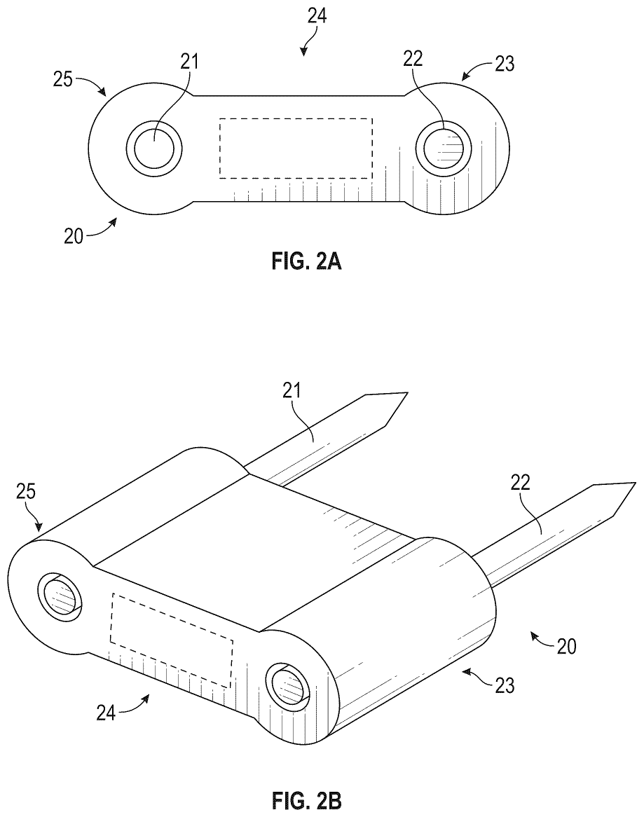

The following exemplary SI joint stabilizing implants may be used in any of the methods of SI joint stabilization herein. Even if the textual description of an embodiment does not expressly include it, it is understood that features shown in different embodiments may be incorporated together. For example, the implants shown in FIGS. 1A, 1B, 2A and 2B each have an interface member with an annular inner surface that defines a lumen, even if the text does not expressly include this description. Additionally, similar components may be similarly labeled in different embodiments. For example, it is understood that references to elements 10, 20, 30, 40, etc., in the figures may illustrate systems, even if the text related to any particular embodiment is silent with reference to a reference number shown in the figure.

FIGS. 1A and 1B illustrate (dorsal view and perspective view, respectively) an exemplary system 10 that includes SI joint stabilizing implant 14 that includes an ilium guide interface member 18 interfacing, which may be also referred to herein as engaging, with elongate ilium guide 12. Implant 14 includes main body 16, a portion of which is disposed across the joint when the implanted is implanted. The interface member 18 includes a surface 19 that has a configuration, in this example annular, that is sized and configured to interface with the corresponding configuration of elongate ilium guide 12. In these figures, the guide may or may not already be positioned in an ilium, such as at the exemplary general location shown in FIG. 11. The interface between the guide and the interface member of the implant restricts the movement of the implant interface guide member with respect to the elongate ilium guide in one or more directions. In this example, implant may still be rotated relative to the guide. In this embodiment, the guide has a cylindrical configuration, with an annular outer profile in cross section along almost all of its length (except for the distal tip region, which may be configured to penetrate into bone). In figures herein, including FIGS. 1A and 10, “S” refers to sacrum, and “I” refers to ilium. Any of the guides herein may be considered to have a cylindrical configuration along all or substantially all of its length.

Any of the guides herein may include a sharpened or pointed distal end (e.g., as shown in FIGS. 1B, 2B, 3B, etc.) that may be configured to help penetrate into bone, such as iliac bone and/or sacral bone.

FIGS. 2A and 2B illustrate exemplary system 20 that includes SI joint stabilizing implant 24, ilium guide 22, and an optional elongate sacral guide 21. In the figures shown, the ilium guide 22 and the sacral guide 21 may or may not yet be positioned within the ilium and sacrum, respectively. In some methods, the one or more guides may be inserted into bone, and then the implant may be advanced over the guides. In some embodiments, the implant is interfaced with the one or more guides, and subsequently the one or more guides can be inserted into bone. Interfacing the implant to a plurality of guides (in examples with more than one guide) before guide insertion may help prevent the guides from being inserted into bone and spaced apart at positions that prevent the implant from then be interfaced with the guides and successfully advanced along the guides and across the SI joint. Interfacing the implant with the guides first may help the guides being properly spaced apart to accommodate the implant during implantation. In any embodiment herein, an ilium guide may be inserted into iliac bone, followed by interfacing the guide with the implant, followed by interfacing the implant with a sacrum guide, and followed by inserting the sacrum guide into a sacrum. The implant may then be advanced across the SI joint.

Any of the dashed lines herein (such as in FIGS. 1A, 2A, 3A and 4A) in an implant body can indicate an optional bore or opening within a main body portion of the implant, which may extend through distal and proximal implant body surfaces.

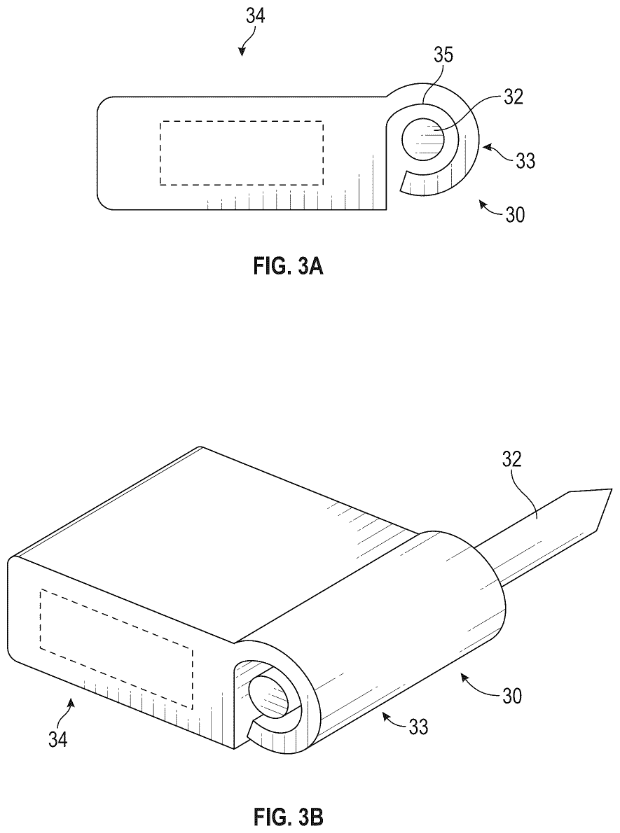

FIGS. 3A and 3B illustrate an exemplary system 30 that includes implant 34, which is configured to interface with guide 32. Implant 34 includes guide interface member 33 that has a surface 35 sized and configured to interface with guide 32, which may be an ilium guide.

Member 33 is in this embodiment curvilinear and has an almost completely annular configuration.

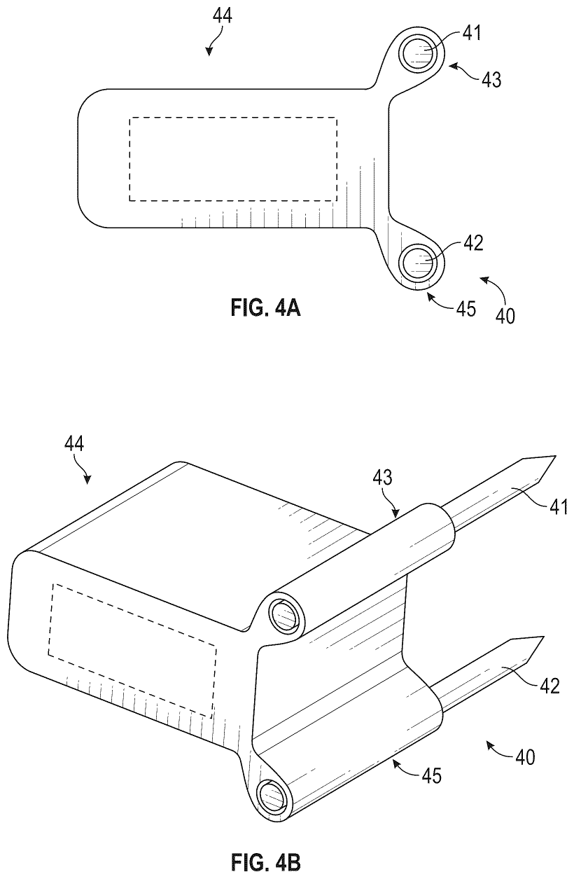

FIGS. 4A and 4B illustrate exemplary system 40 that includes implant 44 with first and second ilium guide interface members 43 and 45, each of which has a surface configured to interface with guide 41 and guide 42, respectively. Members 43 and 45 in this embodiment extend away from the main body region further than the guide interface members in FIGS. 1A, 1B, 2A and 2B, for example.

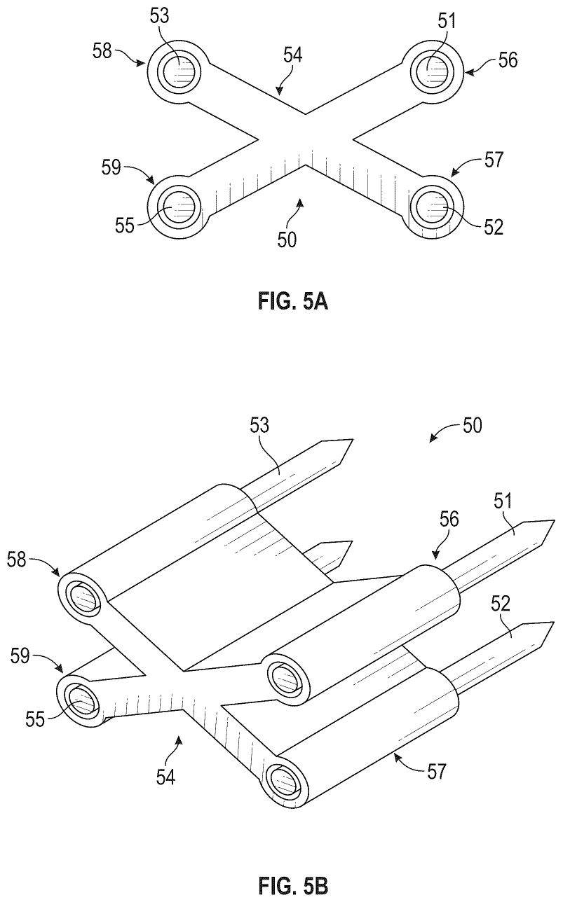

FIGS. 5A and 5B illustrate an exemplary system 50 that includes exemplary implant 54, and exemplary ilium guides 51 and 52 and sacral guides 53 and 55. Implant 50 includes four guide members 56, 57, 58 and 59, each configured to interface with a separate guide. The implant main body has a general “X” or crossing configuration, but could have other main body configurations, such as square, rectangular, oval, etc., and may still have four (or more) guide interface members.

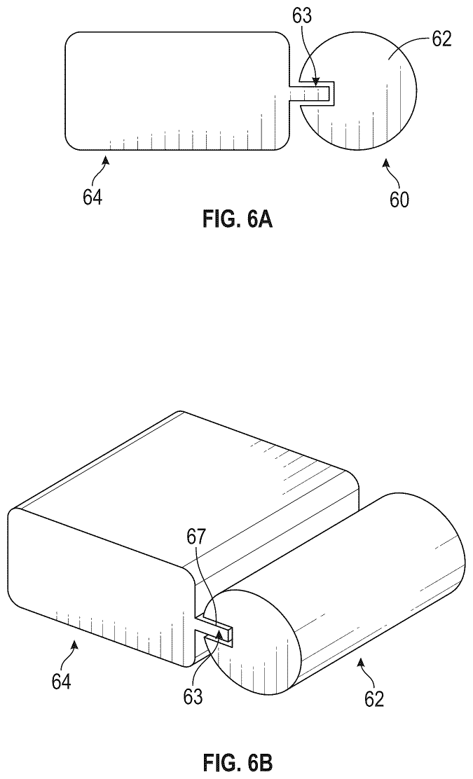

FIGS. 6A and 6B illustrate an exemplary system 60 that includes implant 64 configured to interface with guide 62. In this embodiment guide 62 includes a recessed region that is configured to stably interface with interface member 63 that in this example is a protrusion or extension from a main body region of the implant. This is an example of the implant having a guide interface member that extends within the guide, compared with guide interface members that extend around a portion of the guide, such as in FIGS. 1A-5B. The interface in this embodiment causes guide 62 to act as a guide for implant 64 during implantation.

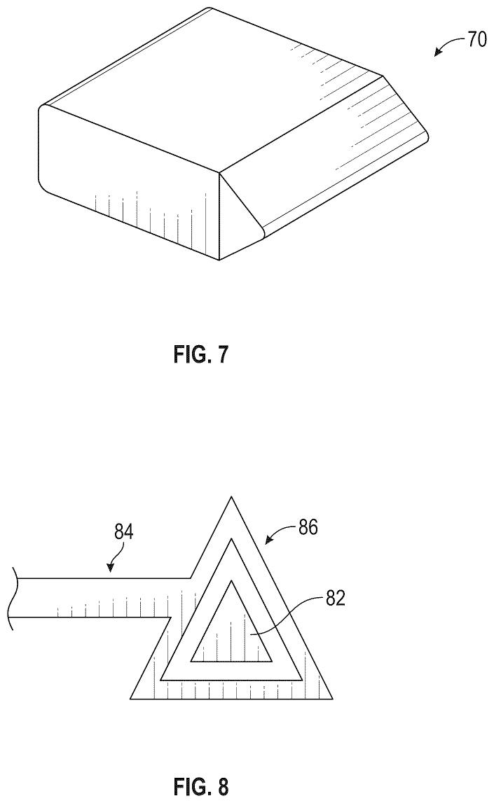

FIG. 7 illustrates an exemplary implant 70 or broach 70 with a sharpened distal end. If used as a broach, the broach 70 may be configured with any of the guide members herein, and in methods of use can be guided over one or more guides (before the implant is implanted) to create a space across the SI joint for the implant. The broach can be removed, and an implant can then be advanced over the guides, which is described in more detail below.

If used as an implant, the implant 70 may be configured with any of the guide members herein (e.g., including a lumen), and in methods of use can be guided over one or more guides to position the implant. The sharpened region of the implant may create the space as well as function as the implant.

FIG. 8 illustrates an exemplary implant 84 that includes guide interface member 86, which is configured to interface with guide 82. In this exemplary embodiment, guide 82 has a triangular configuration (which may have other rectilinear configurations), and member 86 includes an inner surface triangular configuration (which may have other rectilinear configurations), as shown. Implant 84 may also have any number of members 86, each of which can be configured to interface with a different guide.

Any of the implants herein may also have a guide interface member with a first configuration and a second guide interface member with a second configuration different than the first. For example, any of the implants herein may have one or more interface members the same or similar to member 23, the same or similar to member 33, the same or similar to member 63, and/or the same or similar to members 86.

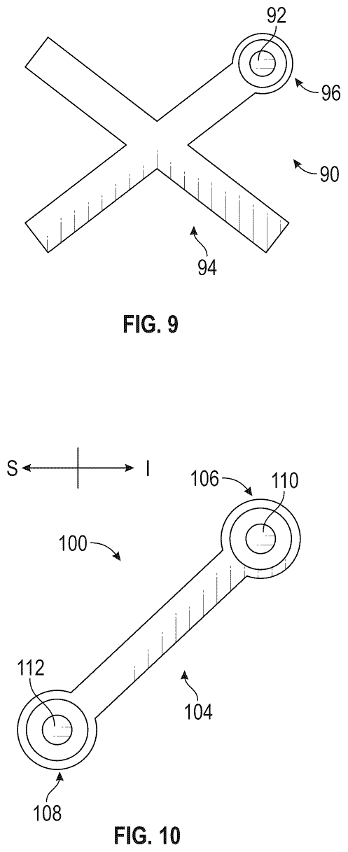

FIG. 9 illustrates an exemplary system 90 that includes implant 94. Implant 94 has a plurality of arms, and not all of the arms include a guide interface member at the respective arm end. In this embodiment only one of the arms has a guide interface member (in this embodiment member 96), but in other embodiments the implant may have any number of members less than the number of arms extending from a main body portion (e.g., two, three, four, etc.)

FIG. 10 illustrates an exemplary system 100 that includes an implant 104 (shown in end view) that includes ilium guide interface member 106 and sacrum guide interface member 108, each of which is configured to interface with guides 110 and 112, respectively. The position shown illustrates an as-implanted positioned across a SI joint, illustrating that any of the implants herein may be implanted with one guide member in one type of bone (e.g., 106) superior to another guide member in a different type of bone (e.g., ilium versus sacrum). For example, guide 110 may be positioned in iliac bone, and guide 112 may be positioned in a sacrum, either inferior to guide 110 as shown, or in other embodiments superior to guide 110, which is not shown, but which would be above guide 110 in FIG. 10.

Any of the implants herein may have one or more surfaces that are configured and adapted to facilitate at least one of bony ingrowth and ongrowth. For example, without limitation, any of the implants herein may include one or more of fenestrations, apertures, porous surfaces, irregular surfaces, etc., such as any that may be described in U.S. Pat. No. 9,044,321, U.S. Published Application 2013/0296953, U.S. Pat. Nos. 9,662,157, 10,166,033, U.S. Published Application 2016/0287171, the disclosures of which are incorporated by reference herein for all purposes.

As is set forth herein, SI joint implants herein may include one or more interface members, which may be configured as lumens or bores. The interface members are generally sized and configured to accommodate passage of one or more guides (such as an ilium guide), which are positioned within one or more of an ilium or a sacrum. In this way, the SI joint implants may be guided by the positioning guides to the intended implantation location across the SI joint without migrating (or at least minimizing migration) away from the denser iliac bone.

In some embodiments, the SI joint implants may include interface members in opposing sides or side regions of the SI joint implant, an example of which is shown in FIGS. 2A and 2B. In this arrangement, the implant is advanced over the guides to position the implant across the SI joint. The guides may be removed after the SI joint implant is delivered to its desired position, leaving the implant implanted across the SI joint.

One aspect of the disclosure is related to methods of positioning a SI joint stabilization implant across a SI joint from a dorsal approach. In these methods, the SI joint implant may be any of the SI joint implants herein. The methods may include advancing an elongate ilium positioning guide from a dorsal starting point, such as starting point 1122 shown in FIG. 11A, and into an ilium of a subject. For example only, FIGS. 2A and 2B illustrate exemplary ilium guide 22, but other types of ilium guides may be positioned from a dorsal approach into an ilium of the subject. FIG. 11A also illustrates a general region 1120 into which any of the ilium guides herein may be started and advanced into an ilium to function as a guide for the SI joint implant. The methods herein may include engaging a guide interface member of the SI joint implant with a positioning guide to restrict movement of the implant with respect to the positioning guide in at least one direction. For example only, FIGS. 1A and 1B illustrate ilium guide interface member 18 of SI joint implant 14, but other interface members herein may be engaged with any of the guides herein to restrict movement of the SI joint implant with respect to the positioning guide in at least one direction. The methods may include, at a time subsequent to the engaging step, advancing the implant across the SI joint while guiding the implant with the positioning guide to implant the implant across the SI joint. The methods further include removing the positioning guide from the ilium and leaving the implant implanted across the SI joint. The methods may include advancing a positioning guide into an ilium between lateral and medial cortical walls of the ilium, descriptions and locations of which are generally known and shown generally in FIGS. 11A and 11B. In these methods, engaging the implant with an ilium positioning guide helps maintain the implantation trajectory and limits the extent to which the implant migrates towards the sacrum while advancing the implant across the SI joint.

Some methods may also include advancing a sacrum positioning guide into a sacrum of the patient, and further engaging a second guide interface member of the implant with the sacrum positioning guide. In these examples, the implant advancing step may occur while also guiding the implant with the sacrum positioning guide. In these examples, the method also includes removing the sacrum positioning guide from the sacrum. Any of the methods herein may include positioning a sacrum positioning guide into a sacrum before or after an ilium positioning guide is positioned in an ilium.

In some methods, prior to implanting the implant, a sharpened broach may be guided over any of the positioning guides herein and advanced towards the SI joint to create a space for the SI joint implant while guiding the broach with the positioning guide. In these examples, the methods include removing the broach to allow dorsal access to the space. The implant may then be advanced over one or more positioning guides as described elsewhere herein.

Depending on the implant being implanted across the SI joint, any of the methods herein may also include positioning a second ilium positioning guide from a dorsal approach into the ilium of a subject. These examples may also include engaging a second guide interface member of the implant with the second ilium positioning guide to further restrict movement of the implant with respect to the second ilium positioning guide in at least one direction.

Depending on the implant being implanted across the SI joint, any of the methods herein may include positioning first and second sacral positioning guides from a dorsal approach into the sacrum of a subject. These examples may also include engaging first and second sacrum guide interface members of the implant with the first and second sacrum positioning guides to further restrict movement of the implant with respect to the first and second sacrum positioning guides in at least one direction.

Any of the individual method steps set forth herein may be combined with any other suitable method step or sequence of steps, unless the disclosure herein indicates to the contrary.

1-22. (canceled)

23. A method of positioning a sacroiliac (“SI”) joint stabilization implant across an SI joint from a dorsal approach, comprising:
advancing an elongate implant positioning guide (“positioning guide”) in a dorsal trajectory into an ilium of a subject;
positioning an inner surface of the SI joint stabilization implant about at an external surface of the positioning guide to restrict movement of the inner surface with respect to the external surface of the positioning guide in at least one direction;
at a time subsequent to the positioning step, advancing the implant across the SI joint while guiding the implant with the positioning guide; and
removing the positioning guide from the ilium and from a position adjacent to the inner surface.

24. The method of claim 23, wherein advancing the positioning guide into the ilium comprises advancing the positioning guide into the ilium between lateral and medial cortical walls of the ilium.

25. The method of claim 24, further comprising viewing a radiographic teardrop view image that shows a teardrop region of the ilium of the subject to help guide the positioning guide into the ilium between the lateral and medial cortical walls of the ilium.

26. The method of claim 23, wherein advancing the positioning guide comprises advancing the positioning guide only into the ilium.

27. The method of claim 23, further comprising:
at a time subsequent to positioning the positioning guide into the ilium, advancing a sacrum positioning guide into a sacrum of the subject;
positioning a second implant inner surface about an external surface of the sacrum positioning guide, wherein the implant advancing step occurs while also guiding the implant with the sacrum positioning guide; and
removing the sacrum positioning guide from the sacrum.

28. The method of claim 27, wherein positioning the second implant inner surface about the external surface of the sacrum positioning guide occurs prior to advancing the sacrum positioning guide into the sacrum of the subject.

29. The method of claim 27, further comprising advancing a second sacrum positioning guide in a dorsal trajectory into the sacrum of a subject, and positioning a third implant inner surface about an external surface of the second sacrum positioning guide to restrict movement of the implant with respect to the second sacrum positioning guide in at least one direction.

30. The method of claim 23, wherein the positioning step limits the amount of implant migration towards a sacrum while advancing the implant across the SI joint.

31. The method of claim 23, further comprising, at a time prior to positioning the inner surface of the SI joint stabilization implant about the external surface of the positioning guide, engaging a sharpened broach with the positioning guide, and advancing the broach towards the SI joint to create a space for the implant while guiding the broach with the positioning guide; and removing the broach to allow dorsal access to the space.

32. The method of claim 23, wherein the implant has a sharpened front section, and wherein advancing the implant into the SI joint comprises advancing the sharpened front section through bone.

33. The method of claim 23, wherein positioning the inner surface of the SI joint stabilization implant about the external surface of the positioning guide comprises positioning an annular inner surface of the implant completely around a proximal region of the positioning guide.

34. The method of claim 23, wherein positioning the inner surface of the SI joint stabilization implant about the external surface of the positioning guide comprises positioning an arcuate inner surface of the SI joint stabilization implant around a proximal region of the positioning guide.

35. The method of claim 23, further comprising advancing a second ilium positioning guide in a dorsal trajectory into the ilium of a subject, and positioning a second inner surface of the SI joint stabilization implant about the second ilium positioning guide to restrict movement of the implant with respect to the second ilium positioning guide in at least one direction.

36. A method of positioning a sacroiliac (“SI”) joint stabilization implant across an SI joint from a dorsal approach, comprising:
advancing an elongate ilium positioning guide in a dorsal trajectory into an ilium of a subject;
advancing an elongate sacrum positioning guide in a dorsal trajectory into a sacrum of the subject;
positioning a first inner surface of the SI joint stabilization implant about an external surface of the ilium positioning guide to restrict movement of the first inner surface with respect to the ilium positioning guide in at least one direction;
positioning a second inner surface of the SI joint stabilization implant about an external surface of the sacrum positioning guide to restrict movement of the second inner surface with respect to the sacrum positioning guide in at least one direction;
at a time subsequent to the positioning steps, advancing the implant across the SI joint while guiding the implant with the ilium positioning guide and with the sacrum positioning guide
removing the ilium positioning guide from the ilium; and
removing the sacrum positioning guide from the sacrum.

37. The method of claim 36, wherein advancing the elongate ilium positioning guide occurs prior in time to advancing the elongate sacrum positioning guide.

38. The method of claim 36, wherein advancing the elongate ilium positioning guide occurs subsequent in time to advancing the elongate sacrum positioning guide.

39. The method of claim 36, wherein advancing the ilium positioning guide into the ilium comprises advancing the positioning guide into the ilium between lateral and medial cortical walls of the ilium.

40. The method of claim 36, wherein positioning the second inner surface of the SI joint stabilization implant about an external surface of the sacrum positioning guide occurs prior in time to advancing the sacrum positioning guide into the sacrum of the subject.

41. The method of claim 36, wherein positioning the first inner surface of the SI joint stabilization implant about the external surface of ilium positioning guide occurs prior in time to advancing the ilium positioning guide into the ilium of the subject.

42. The method of claim 36, further comprising advancing a second sacrum positioning guide from a dorsal approach into the sacrum of a subject, and engaging a third inner surface of the implant about the second sacrum positioning guide to restrict movement of the implant with respect to the second sacrum positioning guide in at least one direction.

43. The method of claim 36, wherein positioning the first inner surface of the SI joint stabilization implant about the external surface of the ilium positioning guide comprises positioning an annular inner surface over a proximal region of the ilium positioning guide.

44. The method of claim 36, wherein positioning the second surface of the SI joint stabilization implant about the external surface of the sacrum positioning guide comprises positioning an annular inner surface of the implant over a proximal region of the sacrum positioning guide.
